(19) United States
(12) Patent Application Publication
(10) Pub. No.: US 2005/0260753 A1
(43) Pub. Date: Nov. 24, 2005

(54) Cross-linked hyaluronic acid-laminin gels and use thereof in cell culture and medical implants

(76) Inventors: Abraham Shahar, Rehovot (IL); Zvi Nevo, Herzlis (IL); Shimon Rochkind, Ramat Aviv Gimel (IL)

Correspondence Address: WINSTON & STRAWN LLP, 1700 K STREET, N.W., WASHINGTON, DC, 20006, US

(21) Appl. No.: 10/669,476

(22) Filed: Sep. 23, 2003

Related U.S. Application Data
(63) Continuation of application No. 10/445,394, filed on May 23, 2003, now abandoned.
Application No. 10/445,394 is a continuation-in-part of application No. 10/437,663, filed on May 13, 2003, now abandoned.
Application No. 10/437,663 is a continuation of application No. PCT/IL01/01050, filed on Nov. 13, 2001.
(60) Provisional application No. 60/248,447, filed on Nov. 14, 2000.

(51) Int. Cl. C12N 5/00; A61K 38/16; C07K 14/47

(52) U.S. Cl. 435/404; 530/395; 514/8

(57) ABSTRACT

The present invention concerns universal biocompatible matrices comprising cross-linked hyaluronic acid-laminin gels useful for clinical applications including as implants, for tissue engineering as well as in biotechnology. The gel matrices according to the present invention may be used clinically either per se or as a cell bearing implant, e.g. stem cells or endothelial cells, containing bioactive molecules or drugs. The gels are particularly useful for use for transplantation or as a coating or a scaffold for use on medical devices including stents.

### CROSS-REFERENCE TO RELATED APPLICATION

This application is a continuation of U.S. application Ser. No. 10/445,394 filed May 23, 2003, an application which is a continuation-in-part of U.S. application Ser. No. 10/437,663 filed May 13, 2003, an application which is a continuation of International Application PCT/IL01/01050 filed Nov. 13, 2001, which application claims the benefit of U.S. provisional application 60/248,447 filed Nov. 14, 2000. This application also claims the benefit of U.S. provisional application 60/382,341 filed May 23, 2002. The entire content of each application is expressly incorporated herein by reference thereto.

### FIELD OF THE INVENTION

The present invention concerns universal biocompatible matrices comprising cross-linked hyaluronic acid-laminin gels, processes of making these gels and uses thereof for clinical applications including as implants for guided tissue regeneration, for tissue engineering and for coating of medical devices, as well as in biotechnology.

### BACKGROUND OF THE INVENTION

The ability to induce and guide tissue regeneration is an unmet medical need, particularly in systems such as the central nervous system and the cardiovascular system where loss of function results in severe debilitation or death.

Neuronal cell death as a result of injury, ischemia or degeneration within the central nervous system (CNS) is generally considered irreversible. Nerve regeneration is largely considered an unattainable goal within the CNS, due to the inability of these cell types to multiply after maturation, which occurs early in life. Axonal injury within the central nervous system is also generally thought to be irreversible when it involves severance of the axons. Various reports of success in nerve regeneration in animal models have not yet led to any satisfactory therapeutic approach to this problem, though it is envisaged that implants or transplants containing viable neurons or their progenitors, possibly derived from human embryonic stem cells, may one day provide an option for attaining CNS regeneration.

The cardiac muscle and cardiovascular system are largely considered to be incapable of regenerating their original structure following myocardial infarct, and therefore arterial occlusion in the heart results in irreparable damage to the cardiac muscle function. One of the therapeutic approaches taken to overcome this pathological phenomenon is the deployment of medical devices called stents to prevent coronary and other vascular system occlusion, though these devices often result in secondary restenosis, due to injury to the endothelial cell layer during introduction of the stent itself.

It is envisaged that these and other major medical problems might be resolved if the implants, transplants or medical devices were provided with a biocompatible scaffold or coating that would enable their integration into the damaged area without evoking secondary damage. Thus, an intracoronary stent may be coated with a biocompatible matrix that would prevent it from eliciting restenosis, or cell bearing medical implants for the CNS might be endowed with the mechanical and biochemical properties that would enable it to survive and propagate as needed.

The attributes of an ideal biocompatible matrix would include the ability to support cell growth either in-vitro or in-vivo, the ability to support the growth of a wide variety of cell types or lineages, the ability to be endowed with varying degrees of flexibility or rigidity required, the ability to have varying degrees of biodegradability, the ability to be introduced into the intended site in vivo without provoking secondary damage, and the ability to serve as a vehicle or reservoir for delivery of drugs or bioactive substances to the desired site of action.

Matrices useful for guided tissue regeneration and/or as biocompatible surfaces useful for tissue culture are well known in the art. These matrices may therefore be considered as substrates for cell growth either in vitro or in vivo. Suitable matrices for tissue growth and/or regeneration include both biodegradable and biostable entities. Among the many candidates that may serve as useful matrices claimed to support tissue growth or regeneration, are included gels, foams, sheets, and numerous porous particulate structures of different forms and shapes.

In many instances the matrix may advantageously be composed of biopolymers, including polypeptides or proteins, as well as various polysaccharides, including proteoglycans and the like. In addition, these biopolymers may be either selected or manipulated in ways that affect their physico-chemical properties. For example biopolymers may be cross-linked either enzymatically, chemically or by other means, thereby providing greater or lesser degrees of rigidity or susceptibility to degradation.

Among the manifold natural polymers which have been disclosed to be useful for tissue engineering or culture, one can enumerate various constituents of the extracellular matrix including fibronectin, various types of collagen, and laminin, as well as keratin, fibrin and fibrinogen, hyaluronic acid, heparin sulfate, chondroitin sulfate and others.

U.S. Pat. Nos. 5,955,438 and 4,971,954 disclose collagen-based matrices cross-linked by sugars, useful for tissue regeneration.

U.S. Pat. No. 5,948,429 discloses methods of making and using biopolymer foams comprising extracellular matrix particulates.

U.S. Pat. Nos. 6,083,383 and 5,411,885 disclose fibrin or fibrinogen glue and methods for using same. U.S. Pat. Nos. 5,279,825 and 5,173,295 disclose a method of enhancing the regeneration of injured nerves and adhesive pharmaceutical formulations comprising fibrin. U.S. Pat. No. 4,642,120 discloses the use of fibrin or fibrinogen glue in promoting repair of defects of cartilage and bone.

U.S. Pat. Nos. 6,124,265 and 6,110,487 disclose methods of making and cross-linking keratin-based films and sheets and of making porous keratin scaffolds and products of same.

Hyaluronic acid (HA) is a naturally occurring high molecular weight polymer belonging to the glycosaminoglycan family, composed of repeating units of glucuronic acid and N-acetyl glucosamine. HA readily forms hydrated gels which serve in vivo as space filling substance. The utility of hyaluronic acid as a beneficial component for supporting tissue growth is well established in the art, as exemplified in U.S. Pat. No. 5,942,499, which discloses methods of promoting bone growth with hyaluronic acid and growth factors. U.S. Pat. Nos. 5,128,326 and 5,783,691 disclose methods of producing and using cross-linked hyaluronans in promoting tissue repair and as reservoirs for bioactive agents including drugs or growth factors

Laminin (LN) is an adhesive glycoprotein of high molecular weight, which is known as a major cell matrix binding component. U.S. Pat. Nos. 4,829,000 and 5,158,874 exemplify uses of gels or matrices comprising laminin.

International Patent Application PCT/IL99/00257 of Shahar et al. (published as WO 99/58042) discloses methods of ameliorating impairments of the central nervous system by culturing neural tissue on a matrix gel composed of hyaluronic acid and laminin. It was previously reported that the combination of HA and LN provides both a flexible elastic bonding and tight rigid bonding cell matrix. Goldman et al. (Ann. N.Y. Acad. Sci. 835, 30-55, 1997) disclosed certain preliminary results using this technique, without providing any details or methods for obtaining these gels.

Nowhere in the background art is it taught or suggested that matrices of hyaluronic acid and laminin are useful for clinical applications in vivo, or that such gels are useful for culture with non-neuronal cell types. Furthermore, the use of these combined HA-LN matrices as a coating for medical devices or in an implant suitable for transplantation has never been disclosed, nor has the use of cross-linking agents to provide stabilization of the gels.

### SUMMARY OF THE INVENTION

The present invention now provides a universal matrix which is biocompatible and affords a convenient environment for cell attachment, growth, differentiation and tissue repair. It also provides a matrix suitable for many different cell types and which may conveniently be used either in vitro or in vivo. A preferred gel matrix is useful for clinical applications due to its unique attributes of fostering tissue regeneration. The unique attribute of elasticity of this gel matrix enables its use both for injection into a cavity or as a coating for a medical device or scaffold.

The matrix gels of the invention comprise Hyaluronic Acid combined with Laminin, designated herein as HA-LN gels. The laminin component stabilizes the cells, provides cell attachment sites and improves cell viability, particularly of cells that are intended for use in tissue regeneration. However, laminin on its own suffers from the drawback that its physical characteristics are inappropriate for use in an implant. The HA component provides the physical attributes that are required to enable the laminin to fulfill its purpose. The combined laminin and HA gels are further stabilized by cross-linking to the desired extent, in order to promote or retard biodegradability, to increase or limit the porosity of the gel, to promote suitable hydrodynamic characteristics, and to achieve other desirable properties as required for the clinical utility of these gels either alone, or in conjunction with medical implants or devices.

Methods of using these gels in vivo in clinical applications are disclosed. The gel matrices according to the present invention may be used clinically for a variety of protocols, whether per se, or as a cell-bearing implant, or as a coating for a medical device or scaffold.

In one preferred embodiment, endothelial cells are grown on the gels, providing a non-thrombogenic, metabolically active surface.

In another preferred embodiment, HA-LN gels are used in conjunction with embryonic or adult stem cells to be selected for differentiation into the cell type of choice.

The gels themselves even when devoid of cells may serve as a vehicle to support cell growth in vivo and as a depot to transport various bioactive high molecular weight substances including but not limited to growth factors, growth inhibitors, adhesive molecules, adhesion inhibitors, and the like or small molecular weight drugs.

The gel matrices according to the present invention may advantageously be used as a substrate suitable for supporting cell selection, cell growth, cell propagation and differentiation in vitro as well as in vivo.

The present invention provides novel compositions and processes for the production of these compositions. Advantageously, during the production of the compositions it is possible to control the viscosity and the degree of elasticity or malleability of the product, as well as other properties of clinical significance including but not limited to biodegradability, porosity and other attributes.

The degree of cross-linking is controlled by selection of a cross-linking agent, by the concentration of the cross linking agent, by the duration of exposure to the agent, by the temperature, and other parameters as are known in the art. Suitable cross-linking reagents include but are not limited to various sugars, enzymatic means, and chemical cross-linking agents including formaldehyde, glutaraldehyde, and other agents as are known in the art. The use of sugars is currently a most preferred embodiment, inasmuch as these cross-linking agents are generally non-toxic. The physiological levels of sugars present in tissue culture medium may suffice to effect cross-linking though at a very slow rate compared to that achieved by the addition of super-physiological levels of sugars.

The gel matrices according to the invention comprise hyaluronic acid in the range of about 0.05% to about 5% (w/v) and laminin in the range of about 0.005% to 0.5% (w/v). More preferable ranges of hyaluronic acid are from about 0.2 to about 3%. Most preferably hyaluronic acid comprises about 0.5 to 2% of the gels. More preferable ranges of laminin are 0.05% to 0.2%.

Viscosity of the gel matrices in accordance with the intended utility may range from 4 to 48 centipoise. Currently most preferred viscosities range form 20 to 25 centipoise.

The present invention also provides method for the addition of further active ingredients to matrices comprising hyaluronic acid and laminin, including but not limited to hormones, growth factors, growth inhibitors, adhesion factors, adhesion inhibitors, anti-fibrotic agents, agents that prevent restenosis, anti-coagulants, coagulation promoting agents, anti-oxidants, anti-inflammatory agents and the like. These optional additives may be incorporated in such a manner to provide for desired pharmacokinetic profiles. Within the scope of the present invention there are provided methods of using the HA-LN gels for sustained release of bioactive components in vivo. In other instances the additives may be incorporated in such a manner to provide for short-lived optimal local concentrations of the bioactive molecules incorporated therein.

The compositions of the invention may further comprise additional macromolecular structural components including but not limited to additional extracellular matrix components, or natural or synthetic polymers, as are well known in the art. According to certain preferred embodiments it is possible to include synthetic or natural polymers in the form of a plurality of carriers dispersed within the gel. According to other preferred embodiments it is possible to use polymers as a mesh or scaffold within the gel.

The compositions of the invention may further comprise additives including preservatives, antimicrobials, isotonicity agents, buffering agents and the like as are well known in the art.

The physico-chemical parameters of the gel matrix, including but not limited to the physical mechanical properties of these gels may readily be optimized in accordance with the intended use of the gel, and methods are disclosed to provide guidance to the skilled artisan in optimization. The biological parameters of the gels may also be controlled including the cell bearing capacity or cell load of the product. Currently most preferred embodiments comprise cell densities ranging from 10⁵ to 10⁷ cells per ml. of the gel.

Devices comprising the gels of the present invention are disclosed as well as uses of such devices.

According to one particularly preferred embodiment, coronary stents coated with the gels of the invention are provided. The gel adapted for coating these devices may further comprise cells. The devices coated with the gels of the invention may further comprise drugs, including but not limited to growth modulators such as growth inhibitory agents, growth factors or hormones including but not limited to drugs that prevent or diminish restenosis.

### BRIEF DESCRIPTION OF THE DRAWING FIGURES

FIG. 1 (A) A schematic representation of a stent (1) coated in an HA-LN gel. The gel (2) form a tube or a sleeve surrounding the stent (1) embedded within. The gel may have an exterior portion with higher viscosity as an exposed surface (3) and an interior surface (4), surrounding the open lumen (5), which forms upon expansion of the stent. The gel-coated stent is expandable either by a balloon that is placed within the crimped lumen or by other means such as Nitinol (nickel-titanium) shape memory alloy.

(B) A schematic representation of a scaffold (1) for implantation within the spinal column comprising gel (2) with embedded polymer or metal mesh (3), having a cylindrical shape with an internal open lumen (5). The cylinder may be perforated or pre-cut to open along one side (4) to enable wrapping it around the spinal cord for example.

FIG. 2 Same nerve cell from rat embryonic dissociated brain, 6 days in stationary culture, exhibiting neuronal fibers, visualized by phase-contrast microscopy (a) or by fluorescent microtubule associated protein-2 (MAP2) staining (b).

FIG. 3 Dissociated brain rat embryonic sub-cultures, 22 divisions. (a) Fluorescent glial fibrilary associated protein (GFAP) staining of astrocytes. (b) Fluorescent MAP2 staining of a neuron.

FIG. 4 Sprouting of rat embryonic dissociated brain cells 24 hours on DE53 MCs in HA-LN gel in the absence (a) or presence of 100 nM pifithrin-α (b).

FIG. 5 Sprouting of rat embryonic brain cells grown for 12 days in HA-LN gel without any additives (a), in the presence of 330 μM ascorbic acid (b) and in the presence of 10 μM N-acetyl-L cystein (NAC) (c).

FIG. 6 Growth of rat embryonic brain cells in HA-LN-gel. (a) Control (after 14 days in gel). (b) Cells treated with low power laser irradiation (LPLI) for 1 min, 50 mW (after 14 days gel). (c) Cells treated with low power laser irradiation (LPLI) for 4 min, 50 mW (after 20 days in gel).

FIG. 7 Elongated bundles of neuronal fibers of rat embryonic dissociated brain cells grown for 6 days in HA-LN gel in the absence (a) and presence of 200 nM pifithrin a (b).

FIG. 8 Human Endothelial cells extracted from pre-term umbilical cords. (a) Cells from 18 weeks abortion cord (3 days in vitro). (b) Cells from 21 weeks cord (10 days in vitro after 3 passages). (c) Cells from 19 weeks cord (2 days in vitro). Endothelial cells undergoing mitosis are marked with an arrow.

FIG. 9 Human endothelial cells extracted from adult veins. (a) Islands of cells after plating and wash 93 days in vitro). (b) Spreading of cells from an island and multiplications (10 days in vitro). (c) Exponential endothelial cells growth after leg phase (27 days in vitro after 3 passages).

FIG. 10 Human endothelial cells seeded on or in various substrates and milieus. (a) On plastic. (b) On gelatin. (c) On HA. (d) In HA. (e) On gelatin and 24 hours later covered with HA. (f) On laminin

FIG. 11 Human endothelial cells seeded on or in HA-LN gel. (a) On HA-LN gel. (b) In HA-LN gel. (c) On gelatin and 24 hours later covered with HA-LN gel.

FIG. 12 Human endothelial cells seeded on or in various substrates and milieus. (a) On gelatin coating. (b) On HA-LN-gel coating. (c) In HA-LN-gel. (d) Twice the amount of cells HA-LN-gel

### DETAILED DESCRIPTION OF THE PREFERRED EMBODIMENTS

It is now disclosed according to the principles of the present invention that cross-linked gels comprising hyaluronic acid combined with laminin have unique attributes that make them suitable for a very wide variety of cell types as well as for use as implants or for coating medical devices intended for implantation into a human subject. The gels of the invention may be adapted for use either with or without cells, for injection, for filling a cavity or for coating a medical device or scaffold, and for many other applications either in vivo or in vitro. It is explicitly understood that the gels are suitable for the culture of cells in a two dimensional as well as a three dimensional manner at varying cell densities.

The unique advantages of the present gels over many other matrices known in the art include their ability to support cell growth, particularly of cell types for which satisfactory growth is not readily achieved, as exemplified for neural cell types, as a non-limitative example. Importantly, the gels of the present invention lend themselves to differential support of cell types, so that it is possible to maintain or propagate the desired cell types while suppressing undesired cell types. This property may be enhanced by specific additives, selected for their known ability to promote or suppress cell growth in a cell lineage specific manner. These additives include growth factors, hormones, growth modulators, and drugs.

The gels of the present invention will be cross-linked, preferably by the use of sugars, or enzymes though the desired extent of cross-linking may be accomplished by any means known in the art. The additives that are to be incorporated into the gel matrix may also be cross-linked to the gel components or otherwise entrapped, to control their release at an appropriate rate. Thus the release of agents incorporated into the gels may be controlled, as well as the biodegradation of the whole implant or gel coating.

(a) Development of the HA-LN Gel for Neuronal Cell Types.

The extracellular matrix (ECM) components of tissues have an important role in directing and regulating the cellular activities. In order to simulate the ECM environment for neuronal and glial cultures the current described procedures were first developed.

The methodologies for the maintenance, growth and differentiation of neuronal cultures are known to be most sophisticated. Therefore, an ECM milieu that mimics the in vivo substrate and requirements of neuronal cells is most desirable. Therefore, an ECM milieu which mimics the in vivo situation of neuronal cells is most desirable.

Tissue culture methods have gained attention as a substitute for the use of in vivo animal models. One direction was devoted to the creation and simulation in vitro of the in vivo environment, nature and composition of the extracellular matrix (ECM) for the cultured cells or explants. It is now disclosed that based on a thorough review of the role of ECM substances in the development of the nervous system, two major components, namely Hyaluronic acid (HA) and Laminin (LN), have emerged as essential candidates specially for neuronal and glial cell cultures.

The combination of HA and LN into one viscous adhesive gel (HA-LN gel) has provided a biomatrix for growing neuronal cells and explants the derived from both the central and the peripheral nervous systems. The combination of HA and LN, which are major components of the ECM have been introduced by the inventors as substrates for growing neuronal cells and explants derived from both the central and peripheral nervous systems.

It is now disclosed for the first time that in addition to providing a substrate matrix in vitro, the HA-LN gel serves as a highly advantageous biocompatible delivery vehicle for implantation.

(b) Development of HA-LN Gels for Additional Cell Types

It is now disclosed for the first time that the in addition to providing a matrix well suited for neuronal cell types the gels of the invention are highly suitable for a wide variety of cell types. As will be exemplified herein below, these gels overcome many drawbacks of existing cell culture technologies.

In addition to neural and endothelial cells the HA-LN gel provides an appropriate substrate for growing primary and secondary cultures of tissue explants and cells, as well as for established cell lines and transformed or bioengineered cells in culture.

Thus, by way of example, these gels have now been found to be adapted to the purpose of cultures of endothelial cell types, epithelial cell types, bone marrow stem cells, embryonic stem cells, progenitor cells derived from embryonic stem cells, beta cells, chondrocytes, and many other cell types for which it has proven difficult to obtain a suitable milieu.

(c) Development of HA-LN Gels for Coating Medical Devices

Due to the advantageous properties of the gels it is now disclosed that these gels are particularly useful to coat medical devices thereby improving the biocompatibility of a variety of medical implants whether inert or cell bearing. According to one preferred embodiment, the cells born by the gel-coated implants are endothelial cells.

According to one currently more preferred embodiment, the gels of the invention are used to coat a stent, either with or without cells. It has now been found that the gels used to coat stents may further advantageously serve as a milieu comprising endothelial cells. These endothelial cells may suppress or diminish restenosis, which often occurs following the placement of the stent. The endothelial cells may be obtained from human umbilical cord or other compatible sources, including but not limited to human embryonic stem cells. A unique advantage of the gels of the invention for this purpose is that they are flexible, pliable and elastic and may be distended in order to allow the deployment of the stent at the desired vascular site.

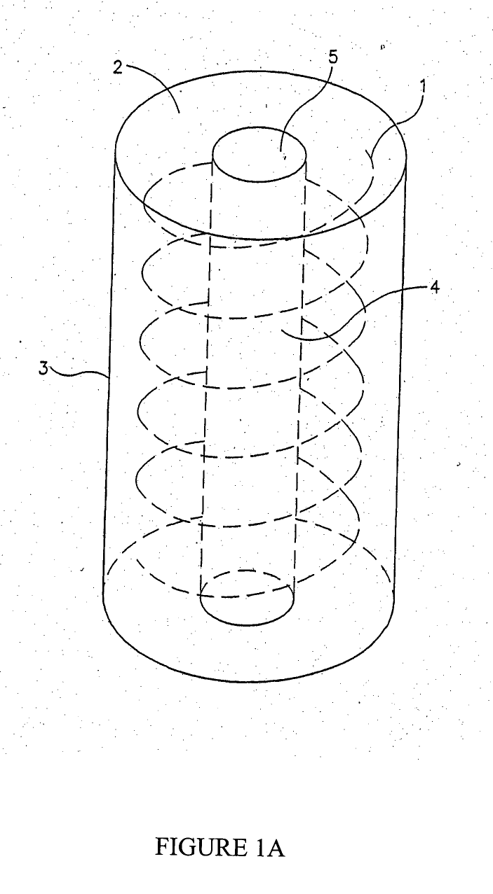

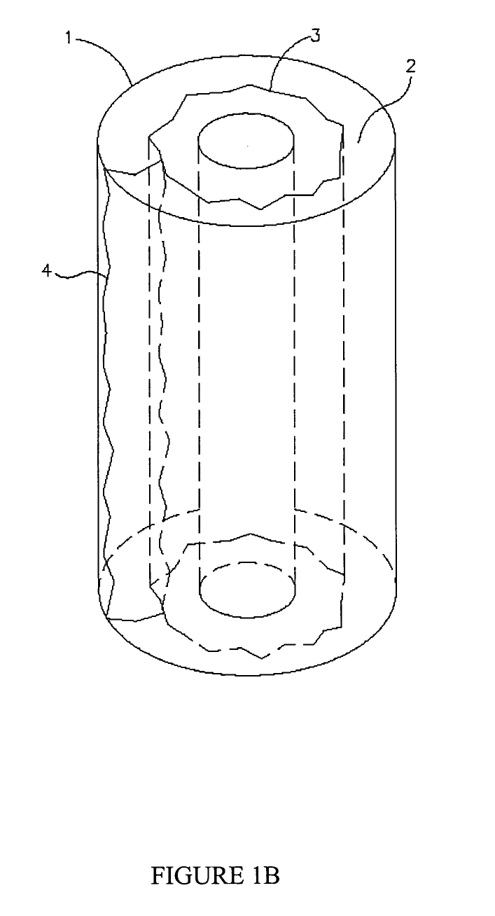

Gel can be applied in various ways, directly on the stent, or as an elastic, expandable tube covering the stent device scaffold, as shown schematically in FIG. 1A. Viscosity of the gel can be uniform or can vary from the internal side of the tube to its external side that will be in contact with the blood vessels. The gel and any carrier materials encasing the stent scaffold can be made to have various porosities, as well as different biodegradation rates. These two features allow a controlled release rate of bioactive compounds or other additives from within the gel matrices. The release rate may be at a slow steady rate, or in certain circumstances it may be designed to produce an initial burst release.

Drug eluting polymer coatings for stents have been reported (e.g., Tao Peng et al., 1996; EP-701802) which teach polymer stents that can incorporate or bind drugs for later local controlled delivery at the target site that would inhibit thrombus formation an neointimal proliferation. Local administration of various drugs including urokinase, heparin, taxol, and hirudin peptide have been proposed to prevent thrombosis and restenosis.

The gel by itself, or when coated on a scaffold of a vascular stent or in other applications, can serve as a physical buffer having advantageous properties. For example the gel as a coating on the stent may provide a physical buffer that will prevent damage to the endothelial surface of the blood vessel upon placement of the stent.

(d) Detailed Features of the ECM-Gel Components:
1) Hyaluronic Acid (HA):

HA was introduced as a viscous growth permissive milieu (Robinson et. al. 1990). It is a natural occurring high molecular weight polymer (2.5-3.0×10⁶ Dalton) that belongs to the glycosaminoglycan family. Compound of this family are composed of repeating units of uronic acid (glucuronic acid) and N-acetyl hexosamine (N-acetylglucosamine). In a hydrophilic environment, HA imbibes large amounts of water molecules (Laurent 1964; Ruohslahti 1988; Preston et. al. 1965). Under these conditions HA is forming hydrated gels of a manipulated viscosity dependency. These gels are serving in vivo as a space filling substance (Longaker et. al. 1989). During the early developmental stages of a fetus, HA is a major component of the ECM, which is considered an optimal environment for repair regeneration and wound healing. Later in life HA is found in joints, synovial fluids, in the genital tract and in other tissue matrices, such as cartilage and the nervous system (Gahwiler 1984; Yasuhara et. al. 1994). HA is the ligand of many cell surface receptors and cell membrane proteins (Knudson and Knudson 1993). Further advantages related to HA in vivo are: a non-antigenic substance, humidity holder, elastic rheological lubricant, antiangiogenic agent, and an antioxidant (Balazs and Denlinger 1988; Toole 1982).

In vitro, HA serves as a growing milieu, traps ions, cells and growth factors and helps cell motility, as disclosed for example by one of the present inventors in Israeli Patent 91080. In addition, it has been reported to modulate neuronal migration and neurite outgrowth (Kapfhammer and Schwab 1992; Thomas et. al. 1993). HA is a biodegradable molecule sensitive to degrading enzymes, such as hyaluronidases and chondroitinases.
2) Laminin (LN):

The LN are well defined family of glycoproteins that provide an integral part of the structural and functional scaffolding of almost every mammalian tissue, e.g. basement membranes conveying messages to cells. The LN is an adhesive glycoprotein-ligand composed of three sub-units with a molecular weight of 900,000 Daltons. Laminins possess the RGDS (Arg-Gly-Asp-Ser) sequence recognized by the transmembranal structure of the most common integrin (α₅β₁). LN-integrin is known as a major cell-matrix binding structure. Each LN is a heterotrimer assembled from alpha, beta and gamma chain subunits, secreted and incorporated into cell-associated extra-cellular matrices. The different types of LN (currently there are 12 known types) can self-assemble, bind to other matrix macromolecules, and/or interact with cells via integrin receptors, dystroglycon or any other even non-integrin receptors. LNs critically contribute to cell differentiation, cell growth, cell shape, migration and movement, preservation of cell-tissue phenotype and elongate tissue survival. The different LNs have been found to be involved in coordinating and guiding many developmental roles in diverse cell types and cell migration toward their final sites during organogenesis (Colognato & Yurchenko, 2000). To date, twelve isoforms of LN have been identified assembled from a repertoire of five alpha chains, three beta chains and two gamma chains (Miner & Patton, 1999). A better understanding of the LNs could provide a basis of therapy to several major pathologies, e.g. merosin-deficient congenital muscular dystrophy.

In summation the LNs display a remarkable repertoire of functions, most importantly as structural elements. Furthermore the LNs serve as signaling molecules providing the cells with diverse information by interacting with cell surface components belonging to the adhesive molecules such as the integrins, connecting the cytoskeleton and the cellular biosynthetic machinery of cells. In developing migrating neurons recently a new cell adhesion molecule designated gicerin was discovered which displays binding activity to neurite outgrowth factor (NOF), which belongs to the LN family (Tairu, 1999). Gicerin promotes neurite extension during embryonic development and participates in the formation and histogenesis of neural tissue later in life. Gicerin is expressed during regeneration in other tissue than the nervous system as well (Tairu, 1999).

LNs are potent stimulators of neurite adhesion and outgrowth in vitro, reflecting an in vivo role in acceleration of axon outgrowth (Powell & Kleinman, 1997).

LN has proven to be an influential glycoprotein of the ECM, which guides and promotes the differentiation and growth of neurons and growth cone behavior (Luckenbill & Edds, 1997). Changes of cell surface integrin expression regulate as well neuronal adhesion and neurite outgrowth (Condic & Letoumeau, 1997). Neuronal LN receptors play as well a key role in neuronal outgrowth (Edgar, 1989; Mecham, 1991). Manipulations of the LNs and LN receptors activity can be obtained by using antibodies against the ligands (laminins) or their receptors which finally determine axonal regeneration (Ivins et. al., 1998), or the neurite outgrowth domain of LN (Liesi et al., 1992). A motor neuron-selective adhesion site on LN receptor acts to inhibit neurite outgrowth (Hunter et al., 1991).

(e) Features of the Gel

It appears that the combination of HA and LN provides both a flexible, elastic bonding and a tight, rigid bonding of cell-matrix.

It is clearly understood according to the principles of the invention that any HA and any LN may be used to prepare the gels of the invention. Hyaluronic acid may be used in its native form, as an uncrosslinked form, or as one of the many chemically modified hyaluronic acid derivatives that are known in the art including but not limited to cross-linked hyaluronans.

Further chemical treatments of the gel mixtures include cross-linking by sugars or additional cross-linking agents or adhesive substances. For instance, by way of a non-limitative example, according to one currently preferred embodiment a solution of sugars including but not limited to one percent D-ribose, D-xylose or any other sugar may be incubated for approximately 24 hrs. in the cold (4° C.) with the gels. The uncoupled sugars are rinsed off the gel prior to use. Small amounts of albumin 0.01-0.1% may be optionally added for improving the gel features, providing additional groups participating in cross-linking.

While it is possible to use other cross-linking agents or enzymatic processes (by way of non-limitative example including factor 13 or lysyl oxidase) to obtain the cross-linked gels, the use of sugars for cross-linking is particularly advantageous due to the non-toxic nature of these naturally occurring agents. The non-toxic nature of the cross-linking agents, and the resultant increase in the molecular weight of the product, stabilizes the gel, and improves the end product.

Cross-linking also serves as a means for converting the gels to a reservoir or depot of additives including high molecular weight cell adhesion molecules, cell growth factors and any other suitable additives. These biomatrix products are viscous, adhesive, highly hydrated formulations simulating the natural extracellular environment and therefore highly biocompatible and conducive for cell growth. Optimization of the matrices includes selection of process parameters to include suitable ranges of the two main components. The composition will affect the rigidity or viscosity of the resultant mixture obtained. Rigid gels may be more suitable for implanting as a molded or shaped implant within an aperture to be filled, while other clinical applications will require the introduction of the matrix as a less rigid, i.e., more fluid or elastic, moldable implant or coating.

Importantly, further ingredients may be used to alter the intrinsic properties of the essential components. By way of a non-limitative example, it may be advantageous to include particulate carriers to which cells may adhere within the gel matrix.

It is now disclosed that it is advantageous to cross-link various groups of the matrix components. The simplest kind of cross-linking bonds are created by sugars including monosaccharides, such as hexoses or pentoses, which bind to free amino groups. Enzymatic bonding of monoamine oxidases (e.g., Factor X111 and lysyl oxidase) creates free aldehydes from free amino groups. These aldehydes, as well as the sugar aldehydes like the reducing end of carbohydrates, e.g., hyaluronic acid, can create an aldol condensation and a Schiff-base product covalently cross-linked. Thus each HA can create one bond with its reducing end residue and many interactions with the hydroxyl groups. Free aldehydes and free amino groups can further react and form crosslinked bonds.

One major attribute of the HA-LN gels is the ability to formulate gels of a desired viscosity or rigidity depending on the concentration of HA and LN as well as the use of cross linking agents and the like.

The structure and the biodegradability of the HA-LN gel may be further modulated by coupling-bonding of low molecular polymers (5-25 kDa) exemplified by, but not limited to, dextran sulfate.

The gel matrices according to the invention comprise hyaluronic acid in the range of about 0.05% to about 5% (w/v) and laminin in the range of about 0.005% to about 0.5% (w/v). More preferable ranges of hyaluronic acid are from about 0.1% to 2%. The selection of the preferable ranges depends on the intended use.

More preferable ranges of laminin are from about 0.05% to 0.2%. The selection of the preferable ranges depends on the intended use.

Viscosity of the gel matrices in accordance with the intended utility may range from 4 to 48 centipoise.

In one currently exemplified preferred embodiment the combined gel comprises 1% hyaluronic acid (as sodium hyaluronate) and 0.01% laminin.

(f) Gel Formulation

The HA-LN gel was developed as a substrate for culturing neuronal-glial cells for implantation. Further extensions and improvements of the HALN-gel for both in vitro and in vivo usages of stimulating neuronal outgrowth are now disclosed along the following lines: The hyaluronic acid (HA) component will be examined as to its optimal molecular weight, concentration, viscosity and possible modifications of the active groups (e.g., hydroxyl to benzyl or other substituent groups as are known in the art).

The second component laminin (LN) may be any one of the twelve types of laminin. According to one currently preferred embodiment laminin-1 is conveniently used. This type of laminin may be replaced with isolated fragments of laminin or laminin derived peptides which retain the desired biological activity as substrate for cell binding. Furthermore, cross-linking between the two components will be induced by any suitable means as are known in the art, preferably using sugar molecules. The interacting outcome will be confirmed by any suitable means as are known in the art including but not limited to crystallographic analysis.

Furthermore, enrichment of the HA-LN gel by additional bioactive molecules in encompassed within the scope of the present invention. Examples of such bioactive components include other extra-cellular matrix (ECM) components (e.g. fibronectin, collagen or the like), adhesive molecules (e.g. integrins including but not limited to nidogen, CD-44, gicerin, dystroglycan, etc.), growth factors (e.g. IGF-I, bFGF, EGF, BDNF, PDGF, NGF etc.), hormones (e.g. estrogen, testosterone etc.), gluing elements (e.g. fibrin or fibrinogen, thrombin, etc.) antioxidants and enzymes to solubilize scar tissue after operations performed in the peripheral nervous system (PNS) and central nervous system (CNS). Non-limitative examples of such enzymes include, but are not limited to trypsin, papain or proteases of plant origin etc.).

The formulation of the gel is highly variable composing specific mixtures creating a spectrum of gels to adjust to the use of a variety of cell type cultures, various tissue implants and various ways of applications for a variety of functions.

Specific combinations of gel components have been developed for a variety of purposes.

HA-LN gels are used as milieu for embedding neuronal composite implants grown on appropriate scaffolds (e.g. pre-treated embryonic spleen tissue used as scaffold for neuronal cells) for transplantation into injured sites of brain and spinal cord tissue.

The use of a HA-LN gel as an implant intended for use as a sheath for guided tissue regeneration in the spinal cord is depicted schematically in FIG. 1B.

Using neuronal cells embedded in HA-LN-gel for filling (either by surgical intervention or also by injection) post-traumatic or post-operative cysts resulting for example from injury, hematomas, or tumor removal.

Combining the HA-LN-gel with additional factors including but not limited to: coagulative factors, anti-fibrotic agents, growth factors, or proteolytic enzymes which might lead respectively to promotion of hemostasis, the solubilization of scar tissue, and enhancing axonal regeneration.

In one currently preferred embodiment, HA-LN gels are used in conjugation with neuroprotective agents, exemplified by, but not limited to ascorbic acid (AA), N-acetyl-L-cystein (NAC) and pifithrin-α.

HA-LN gel matrices are also used in conjunction with embryonal stem cells to be selected for differentiation into the cell type of choice.

Further use of the HA-LN gels is in conjunction with endothelial cells. According to one currently preferred embodiment, composite implants made of HA-LN gels, as well as medical devises covered with the gel, can be successfully covered with endothelial cells to improve their function.

It must be stressed that gels, according to one currently most preferred embodiment of the present invention, have been optimized for use in conjunction with neural cell types, suitable for use in ameliorating deficits and defects in the central nervous system.

According to an alternative most preferred embodiment the gels are used in conjunction with medical devices in the vascular system in general and the cardiovascular system in particular.

The skilled artisan will appreciate the general applicability of the methods disclosed herein. The availability of human cloned cell lines, pluripotent embryonic stem cells, autologous cells, bioengineered cells comprising inserted genes or antisense moieties and additional cell types will lend themselves to use with the methods and compositions of the present invention. Thus, for the sake of example, human embryonic stem cells may be selected or activated to differentiate into any desired cell type suitable for transplantation utilizing the gel matrices of the present invention.

(g) Preferred Uses of the Product

The HA-LN-gel product can be used for the following purposes:
1) Tissue Culture:

An adhesive biological environment that provides an optimal milieu for the anchorage of cells and tissue slices during cultivation.

The product serves as a reservoir for desired pharmacokinetics of growth factors, hormones, signal molecules, inhibitors of cell growth and any other type of cell growth modulators.

In addition the gel enables absorption of nutritional elements and provides mechanical and biochemical protection of the cultured cells, as well as enabling neutralization of damaging cellular metabolites such as free radicals or the like.
2) Delivery Vehicle for Transplantation:

The product serves as a delivery vehicle for transplantation of implants. The implants may be devoid of viable cells or may be loaded with cells according to the intended medical indication being treated.

For use in the central nervous system the transplants will preferably be cell bearing, while for use in bone or cartilage repair they may be used preferably without cells.

The product may also serves as a delivery vehicle for transplantation of cultured implants. The implants composed of cultured slices or dissociated cells embedded in the gel, not only survive but continue their growth for 48 hours in the gel without requiring any addition of nutrient medium

The gel product serves as a storage depot for pharmacological, enzymatic and other agents and drugs such as inhibitors of neurological scar, promoters of neuronal growth, immunosuppressors, chemotherapeutic agents, anti-adhesion agents, anti-fibrotic agents, and other cell growth modulators as required.
3) Coating of a Medical Device:

Gel can be applied in various ways: directly on the medical device as a coating for an external surface, for coating an aperture or lumen in the device, or as an elastic, expandable tube covering the device which serves as a scaffold. Viscosity of the gel can be uniform or can vary from the internal side of the coating to its external side that will be in contact with the tissues into which the device is implanted. The gel and the carrying scaffold material can be made to have various porosities, as well as different biodegradation rates. In one currently preferred embodiment, gels used for coating an external surface of medical devise bear endothelial cells.

(h) Routes for Clinical Applications of the Gel

The HA-LN gels of the present invention is beneficial for a variety of clinical applications, including the following:
1. Injection of the product into affected area, filling in cavities, cysts, etc.
2. Covering resurfacing affected areas with the product functioning as a biological glue.
3. Placing the product within tubes or capsules which will fill the gaps respectively, in nerve reconnection and transplantation into the central nervous system.
4. Placing the product within or as a coating on the exposed surfaces of stents, for vascular uses and angioplasty, respectively, especially for cardiovascular applications.

(i) Indications for Neural Clinical Applications of the HA-LN-gel.
1. Peripheral nerve, spinal cord and brain injuries and damage.
2. Spinal cord, brain peripheral nerve reconstruction/transplantation.
3. Tissue and drug administration in brain degenerative and demyelinative diseases (Alzheimer, Parkinson's disease, Multiple Sclerosis, etc.).
4. Post-operative or post-traumatic brain or spinal cord cysts.
5. Prevention or decreasing of post-injury or post-surgical scarring.
6. Spinal form of multiple sclerosis.

All the above mention utilization of HA-LN-gel in neuronal cultures and implantation are adjustable to other tissue types for treatment of a wide variety of injuries or disorders. Suitable cell or tissue types for use in conjunction with the matrices of the present invention include but are not limited to endothelial cells, liver, cartilage, bone, heart, spleen, lung, skin and blood vessels.

The principles of the invention, providing HA-LN gels matrices useful as a milieu for cell and tissue culturing and for clinical applications according to the present invention, may be better understood with reference to the following non-limiting examples.

### EXAMPLES

The following examples of certain currently preferred embodiments are provided merely for illustrative purposes and are not to be construed as limitative. The scope of the invention is defined by the claims which follow.

(a) Materials

The HA component was provided by BioTechnology General LTD (Rehovot, Israel). It was examined as to its optimal molecular weight, concentration, viscosity, and possible modifications of the active groups (e.g. hydroxyl to benzyl).

The detailed composition of the HA used contained: 90% sodium hyaluronate; molecular weight (mega Daltons)—2.01; protein (mg/g)—0.2; absorbance at 257 nm (1% solution)—0.02; endotoxin (1% solution) (EU/mg)—<0.125; (non-inflammatory substances).

The HA-gel has a viscosity of dynamic intrinsic viscosity as may be measured by streaming a solution in a capillary of a viscometer at 25° C. and expressed as μ viscosity coefficient in centipoise ranging between 8 to 48 depending on the molecular weight that can range between 2 to 8×10⁶ Daltons. (Bag's HA ranges 2.5 to 3×10⁶ Daltons).

The second component LN is tested and compared with different laminin peptides. The best-characterized LN is LN-1 (composed of 1 alpha, 1 beta and 1 gamma): it promotes neuronal outgrowth in all developmental stages in embryonal and adult neurons. It is believed that LN-1 is a guiding substrate for axons in vivo.

Murine LN-1 used in our experiments was obtained from Sigma.

Furthermore, cross-linking between the two components is induced, preferably using sugar molecules. The interacting outcome is confirmed by any appropriate means including crystallographic analysis.

The formulation of the gel is highly variable composing of specific mixtures creating a spectrum of gels in regards to their composition, physical and biological features. The various gels are adjusted to the use of a variety of cell type cultures, various tissue implants and various intended applications for a variety of functions.

(b) Applications of the HA-LN Gel
1) Growth and Maturation of Neuronal Cultures

The HA-LN-gel of the present invention is an excellent viscous milieu for growing nerve cells in culture. Neuronal factors, adhesive molecules and neuroprotective agents were added to the gel is to obtain their slow release by the gel during cultivation, thus enabling intensive neuronal sprouting, growth and maturation.

Some of these factors, such as brain-derived neurotrophic factors (BDNF) and nerve growth factors (NGF) are essential in the initial stage of cultivation and during the establishment of CNS and PNS cultures. Other factors like insulin-like growth factor-1 (IGF 1) and leukemia inhibitory factor (LIF) are needed for nerve cell survival, maturation and myelin formation in culture.

The antioxidants N-acetyl-L-cysteine (NAC) and ascorbic acid (AA), and the protective compound pifithrin-α were found to be neuroprotective agents (both in vitro and in vivo). Slow release of these agents by the enriched HA-LN-gel is therefore beneficial for the survival, growth and maturation of neurons in culture as well as after implantation.

Low power laser irradiation used in brain and spinal cord transplantation in animals showed positive effects on survival of implant and axonal sprouting. Therefore, we used laser irradiated neuronal cells attached to MCs embedded in HA-LN-gel, as a regenerative source for reconstruction of transected spinal cord.

(c) Enriched HA-LN-gel

The following components were added to the HA-LN-gel to form an enriched gel supporting neuronal cells survival, growth and maturation:
1) Adhesive Molecules:

Laminin (Sigma, L-2020), in the concentration of 20-30 μg/ml. In some control experiments, Laminin was replaced with Fibronectin (Biological Industries Co., 03-090-1) for cultivation of cells from the PNS.
2) Neuronal Survival, Growth and Maturation Factors:

BDNF (Sigma, B-3795 or Peprotech Inc., 450-02)—10 ng/ml.

IGF-I (Sigma, I-1271 or Peprotech Inc., 100-11)—2 ng/ml.

NGF (Sigma, N-6009 or Peprotech Inc., 450-01)—10 ng/ml, was added to spinal cord (SC) and to dorsal root ganglia (DRG) cultures only.

LIF (Sigma, L-5158)—0.5 ng/ml, was added to older cultures (more than 14 divisions).

A mixture of the regular brain medium (see below) with the above-mentioned neuronal growth factors (in a calculated dose to achieve a desired final concentration) was included in the gel. The medium was composed of DMED/F12 (Gibco, 31330-038) supplemented with 6 mg % glucose (Sigma, G-8769), 2 mM glutamine (Gibco, 25030-065), antibiotic (Gibco, 15240-062) and 10% fetal bovine serum (Gibco, 10108-165). Cultures were usually seeded in a gel that included 500-600 μl of the factor-medium mixture. When a less viscous gel was required, a larger volume of factor-medium mixture was added.

d) Neural Cultures in the Enriched HA-LN-Gel:
1) Cultivation of Dissociated CNS Cells:

Neuronal cells were seeded in the enriched HA-LN-gel immediately after dissociation. Cells (about 1×10⁶) were embedded in a volume of 1-1.5 ml of the gel. The cell-gel mixture was seeded in 12 well plates or in 35 mm Petri dishes (1×10⁵ /culture) and incubated for 24-48 hours without addition of nutrient medium. Cultures in the gel were then covered with medium (0.5 ml/well or 1 ml/dish), containing neuronal factors.
2) Preparation of Neuronal Stationary Cultures in HA-LN-Gel from Cells Grown in Suspension on DE53 MCs:

After 4-17 days of growth in suspension, floating cell-MC aggregates were collected and transferred (under a stereomicroscope using fine spatula) into the enriched HA-LN-gel (3-4 cell-MC aggregates/well).
3) Production of Sub-Cultures:

The mild enzyme RDB (diluted 1:30 in Hank's balanced salt solution—HBSS) was added to dense old neuronal brain or spinal cord cultures. The cells that were gently detached were collected and re-seeded in the enriched HA-LN-gel as described herein above.
4) Covering of On-Going Neural Cultures with HA-LN-Gel:

Older cultures, or cultures in which nerve-cell-processes were not well attached, were covered with 1-1.5 ml of enriched gel, after removal of the culture medium. Covering with the transparent gel allowed, on one hand, a better observation of neuronal cells within the cell-aggregates, and on the other hand stabilization of processes in the viscous milieu.
5) Fluorescent Staining of Neural Cultures:

In order to characterize neural and glial cells, cultures were incubated with antibodies, which were directed against specific neural and glial markers. Addition of fluorescent secondary antibodies, with different fluorescent dye attached, allowed discrimination between the different cell types. Furthermore, with the aid of an image analyzer, better evaluation of the quantitative distribution of cell population in the culture can be achieved.

After fixation with 3.7% paraformaldehyde (Sigma, P-6148), cultures were incubated with antibodies against the neural cell marker microtubule associated protein—MAP-2 (Sigma, mouse anti rat MAP-2, M-4403) or glial cell marker glial fibrilary associated protein—GFAP (Zymed, rabbit anti-human GFAP, 18-0063).

Cells were then washed and incubated with Texas Red-goat anti mouse IgG (Jackson, 115-075-146) or with Cy™2-goat anti rabbit IgG (Jackson, 115-225-144), respectively. Double staining with two sets of antibodies was performed as well. The stained cultures were observed under fluorescent microscope with suitable filters.
6) Results:

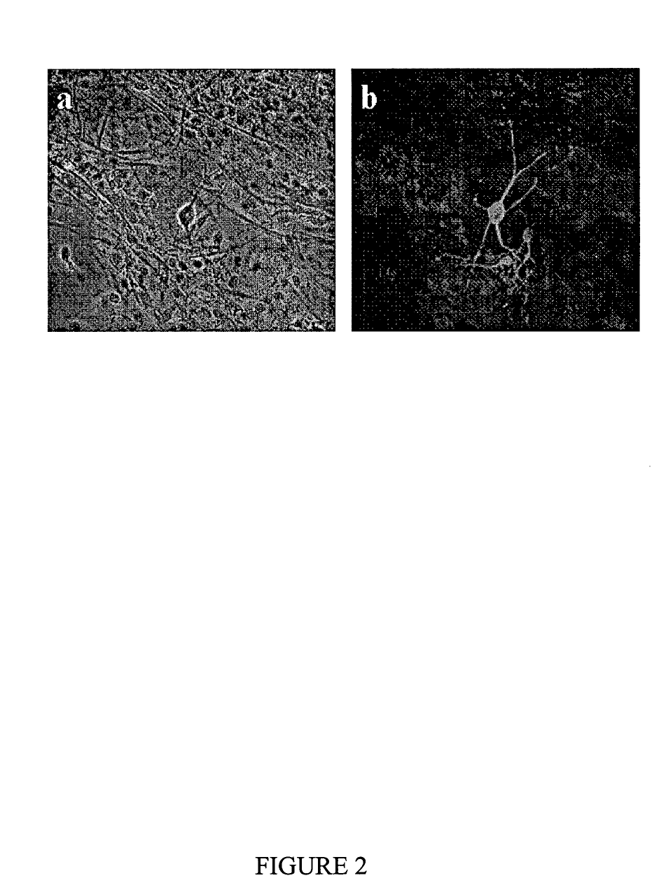

FIG. 2 shows a nerve cell from dissociated embryonic rat brain, after 6 days in stationary culture in HA-LN gels, exhibiting neuronal fibers. MAP-2 fluorescent staining enabled a better characterization of the neural fibers.

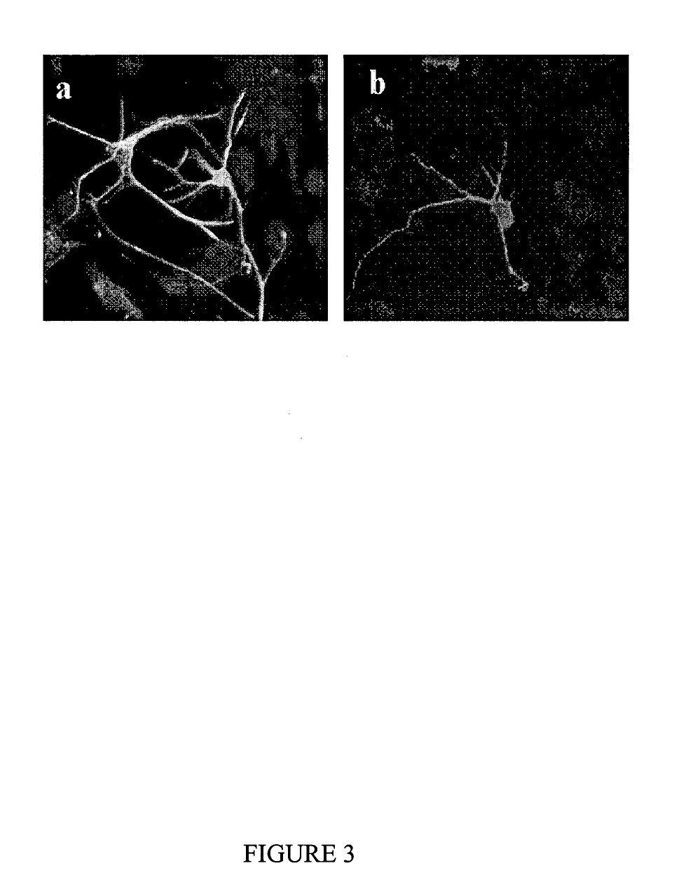

FIG. 3 shows sub-cultures of dissociated embryonic rat brain neuronal cells. Double fluorescent staining with specific antibodies enabled the discrimination between neural cells and glial cells.

e) Antioxidants, Protective Agents Treatments and Low Power Laser Irradiation:
1) Dissociated Nerve Cells Cultured in HA-LN Gel:

Neuronal cells were manually dissociated from brains of 14-16 days rat embryos. Cells were embedded in the HA-LN-gel, which was enriched as described herein above.

NAC and AA were obtained from SIGMA (Cat. No. 616-91-1 and A2218; Lot 108H00575 and 096H02811, respectively). Pifithrin-α, cyclic (Calbiochem, Cat No. 506134, lot:B41595) is a very stable analog of pifithrin-α, with reduced cytotoxicity. NAC, AA and Pifithrin-α cyclic were added to the HA-LN-gel (enriched with neural growth factors) prior to culturing of cells. Culture medium was changed twice a week. All the experiments contained control cultures (without treatment of NAC or AA or Pifithrin-α. 5-7 experiments were performed in 12 wells, in triplicates.
2) Cultivation of Brain Cells on MCs in HA-LN-Gel:

Dissociated brain cells were attached to DE53 cylindrical, positively charged microcarriers (MCs), and suspended in brain medium for 4-17 days (Shahar A 1990 Methods in Neur 2:195-209). The floating cell-MC aggregates were transferred (using fine spatula, under a stereo microscope) into HA-LN-gel enriched with the indicated growth factors or Antioxidants.
3) Laser Irradiation:

For laser irradiation, cells or cell-MC aggregates were concentrated in a small drop (100 μl) of enriched HA-LN-gel enabling exposure of all the cells in the culture to a single irradiation (irradiation area was 30-40 mm²) immediately following seeding.
4) Results:

Ascorbic acid (AA) was applied to the different cell cultures at 1 mM, 330 μM or 100 μM. All concentrations were found to be effective, with the most effective dose found at 330 μM.

For Pifithrin-α, cyclic, application at 100 and 200 nM were found more effective than application at a concentration of 2 μM.

N-acetyl-L-cysteine (NAC) was examined in concentrations of 0.1, 1, and 10 μM. The most effective dose was found to be 10 μM.

Low power laser irradiation (LPLI) (780 nm) was used at the powers of 10 mW, 30 mW, 50 mW, 110 mW, 160 mW and 250 mW. Cultures were irradiated for 1, 3, 4, and 7 min. The most effective treatment was found to be 50 mW for 1 or 4 min.

During one month of daily observations the following differences (which were more pronounced in the stationary MC-cultures) were observed:

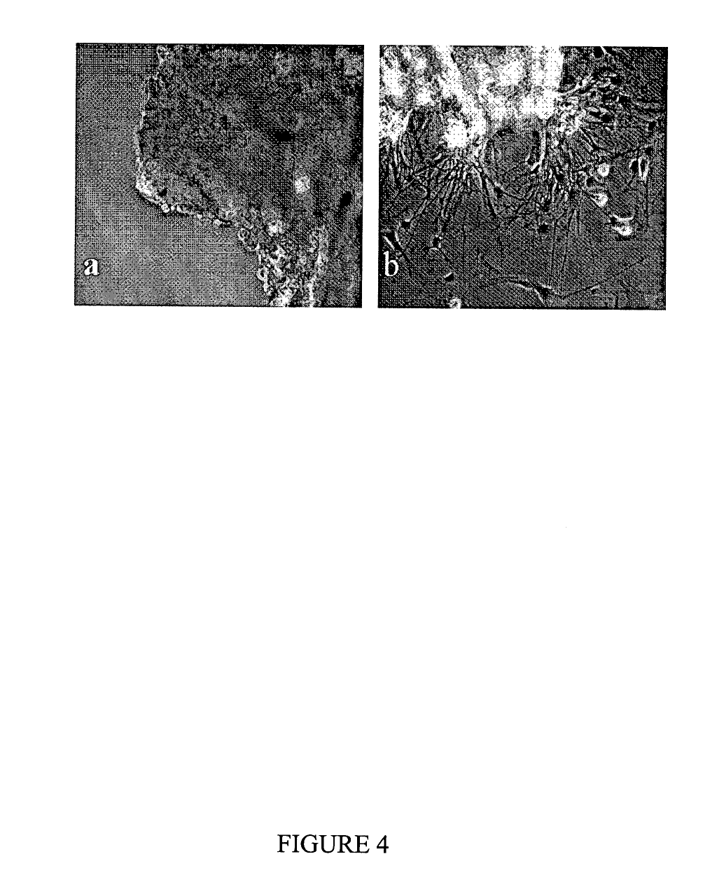

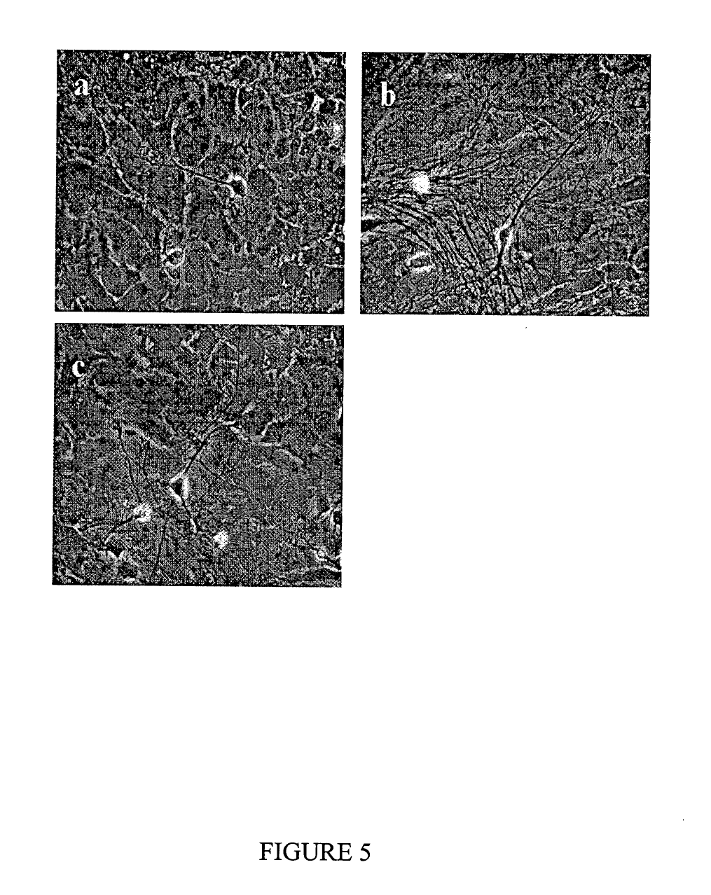

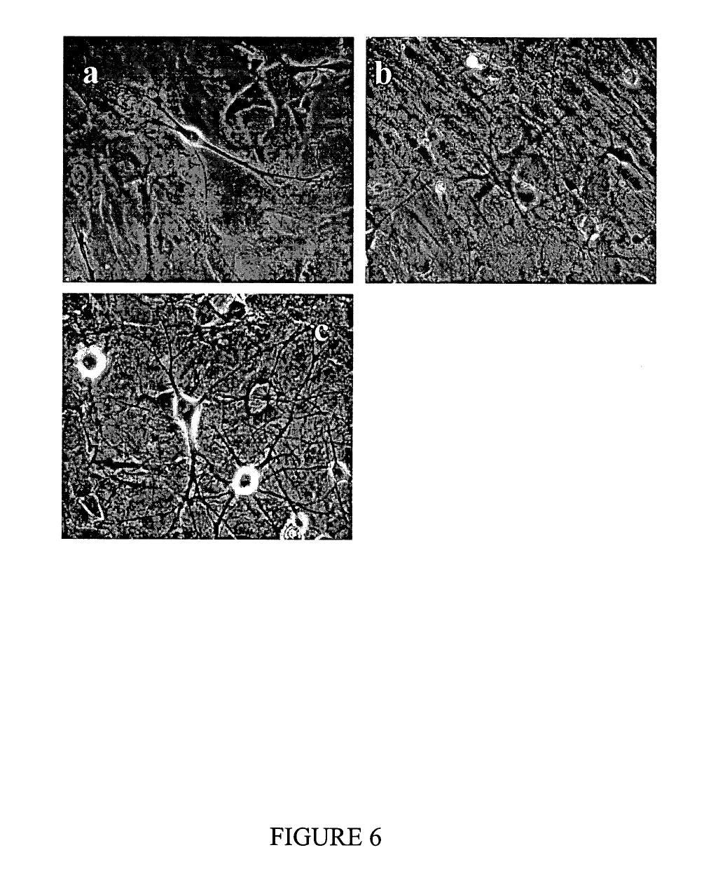

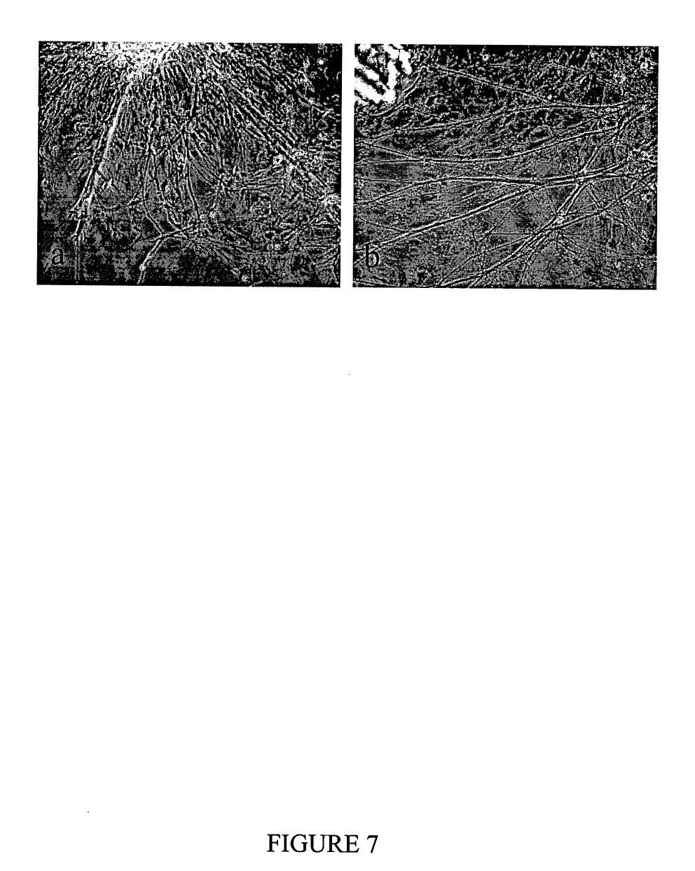

Addition of Pifithrin-α cyclic (FIG. 4), AA, NAC (FIG. 5 (b) and (c) respectively) to the HA-LN-gel, or irradiation with low power laser stimulated intensive sprouting already during the first 24-48 hours of culture in the enriched gel (FIG. 6). Furthermore, during the following weeks the treated cultures contained a higher number of neurons, many of them exhibiting large perikarya. In the treated cultures, cell aggregates extended elongated thick bundles of nerve fibers (FIG. 7).

f) Treatment Procedures with HA-LN Gel Comprising Neuronal Cells
1) Treatment Procedures for Spinal Cord:

This treatment procedure is used in cases of injury, damage, posttraumatic/post surgical cysts or congenital syringomyelia and also in cases of degenerative and demyelinative diseases of spinal cord.

The injection or implantation of HA-LN-gel is introduced by injection or implantation using needle, endoscopic, stereotactic, navigator techniques, etc., or standard surgical approaches with myelotomy.

HA-LN gel is injected or implanted within the cyst cavity or affected area with or without biological materials, such as CNS tissue, stem cells, Schwann cells, growth factors, etc. This procedure would have to be accompanied by microscopic spinal cord untethering at the injury site and expansion duroplasty.
2) Treatment Procedures for Brain:

This treatment procedure is used in cases of injury, damage, posttraumatic/post surgical cysts, cavity, strokes from ischemic and intraparenchymal hemorrhages and also in cases of degenerative (Parkinson's disease and etc.) and demyelinative (multiple sclerosis and amyotrophic lateral sclerosis, etc.) diseases.

The injection or implantation of HALN-gel is introduced by injection or implantation using needle, endoscopic, stereotactic, navigator techniques, etc., or standard surgical approaches.

HA-LN-gel is injected or implanted within the cyst or cavity or affected area with or without biological materials, such as CNS tissue, stem cells, Schwann cells, growth factors, etc.

HA-LN gel is unique in offering the possibility of exposure of explants, neuronal and glial cells, drugs, factors etc., into brain and spinal cord affected area.

In addition, HA-LN gel is playing an important role is in the reconstruction of implants of oligodendrocytes and Schwann cells from fetal and adult origins. These implants, composed of cultured central and peripheral myelin forming cells, are intended for transplantation to cure neuronal disorders resulting in demyelinating effects.

In addition to the CNS the reconstruction of peripheral nerve presents a unique clinical entity on its own.

The peripheral nerve or brachial plexus or cauda equina are exposed and treated microsurgically by external and/or interfascicular neurolysis, or primary sutures, or nerve grafts, scaffolds or tubes. After completion of reconstruction, the exposed peripheral nerve is covered by HA-LN-gel per se or tissue engineered an filled into tubes or scaffolds with or without biological materials, such as Schwann cells, growth factors, drugs, and the like.

g) Growth and Maturation of Endothelial Cells

Endothelial cells are an important component of composite implants and stents as they provide a non-thrombogenic surface, they are metabolically active cells that produce growth factors and cytokines, and they form new blood vessels.

Human endothelial cells were extracted and cultivated from different sources: umbilical veins of pre-term cords; umbilical veins of full-term cords; and adult veins excised during bypass surgery. The cells from all sources exhibited similar morphological characteristics, typical of large blood vessel endothelium. The cells extracted from all the sources were positive in immunofluorescence labeling for classical endothelial markers (vWF and PECAM-1). There were no differences between HUVEC (Human Umbilical Vein Endothelial Cells) extracted from pre- or full-term cords. However, endothelial cells extracted from an adult blood vessel have a leg-period of about 14 days, meaning that they grow much slower at the first 14 days of culture. In addition, variability was found in the viability of cultured cell from different donors of adult veins, which may be correlated with the patient's age and general physiological condition.

Endothelial cells were harvested from human umbilical veins, adult human veins and adult mammary artery, obtained after bypass surgery. Blood vessels were cannulated, rinsed with PBS, then incubated with 0.1% Collagenase (CLS-I; Worthington Biochem. Corp) for 15-20 minutes. The detached cells were plated on gelatin-coated culture plates or flasks. The medium used for endothelial cell cultivation is M-199 with Earle's salts, buffered with 20 mM Hepes and supplemented with 100 μg/ml heparin, 100 μg/ml of endothelial cells growth supplements (extract of bovine brain containing basic and acidic FGF) and 20% FBS (fetal bovine serum). Cells were fed every third day with fresh medium and were split soon after reaching 80% confluence by two minutes digestion with 0.25% trypsin EDTA. Aliquots of endothelial cells from different sources were also frozen in freezing medium (10% DMSO in FBS) and stored in liquid nitrogen.

All cells used in the experiments, or stored frozen, were all up to the fifths passage. Endothelial cells extracted from all sources exhibited typical characteristics such as a large oval nucleus, granular cytoplasm and formed a cobblestone like monolayer. Immunofluorescent staining of cells extracted from several sources with antibodies against specific endothelial markers confirmed their endothelial nature, as elaborated herein.

The different blood vessels used for endothelial cell extraction and cultivation were as follows:

1. Endothelial Cells from Pre-Term Umbilical Vein:

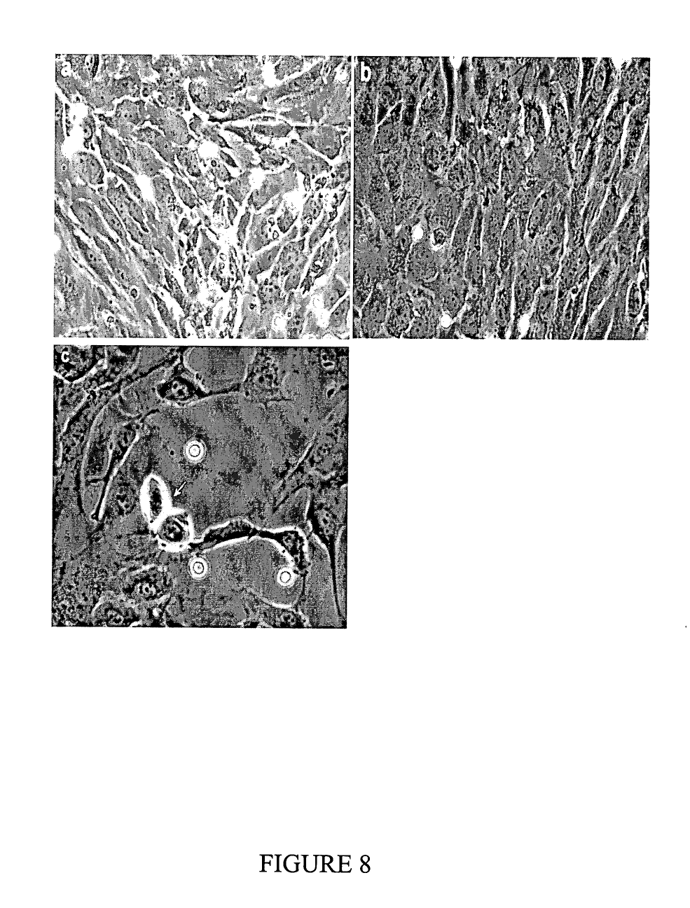

Endothelial cells (EC) extraction from pre-term umbilical cords was performed three times on cords ranging from 18 to 21 weeks abortions. There was no difference in the cell yield or the morphological characteristics of the EC obtained (FIG. 8).

2. Endothelial cells from Adult Veins

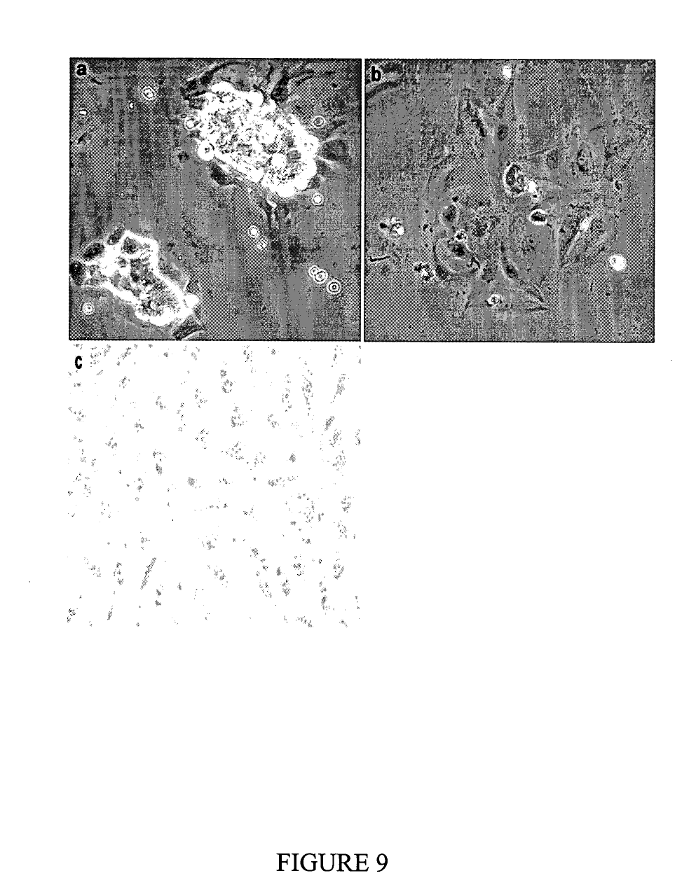

EC were extracted from pieces of veins from adult legs or chest obtained in by-pass operations (FIG. 9). Significant differences were observed in the cell yield and cell viability between vein pieces that had been received up to 24 hours after the operation and cell received after longer periods. Viable EC could not be obtained from vein pieces stored for more than 24 hours. Moreover, EC yield and cell viability decreased from veins obtained from older patients.

In general, EC extracted from adult veins multiplied and grew slower than EC obtained from other sources.

3. Endothelial Cells from Full Term Umbilical Cords

HUVEC (Human Umbilical Vein Endothelial Cells) were extracted from three full term umbilical cords, received from the hospital in one shipment. The cells were cultivated as previously described (Jaffe et al 1972 Circulation 46, 211-253; Jaffe et al 1973 J. Clin Invest 52, 2745-2756).

h) HA-LN-Gel as a Supporting Substrate and Milieu for Endothelial Cells

In order to test the ability of HA-LN-gel to support the attachment and growth of endothelial cells, several experiments were conducted:

Endothelial cells (HE4 split #1), grown on gelatin, were collected by 0.25% trypsin-EDTA digestion. HUVEC were counted and 1.35×10⁶ cells were divided into treatment groups. The cells were seeded in triplicates into 12-well plates at a concentration of 50,000 cells per well. The treatment groups were as follows:
1. Cells seeded on the plastic
2. Cells seeded on gelatin coating
3. Cells seeded on HA (1 ml)+medium (0.5 ml) coating. Each well was coated with ˜0.2 ml
4. Cells (150,000) seeded in HA (1 ml)+medium (0.5 ml). 0.5 ml per well.
5. Cells seeded on gelatin and after attachment (24 hours) covered with HA+medium (as above, coated with 0.5 ml per well)
6. Cells seeded on Laminin coating
7. Cells seeded on HA-LN-gel coating (0.9 ml HA, 0.1 ml Laminin, 0.4 ml medium). Coating was with ˜0.2 ml per well
8. Cells (150,000) seeded in HA-LN-gel (as in g)
9. Cells seeded on gelatin and after attachment (24 hours) covered with HA-LN-gel (as in g above)

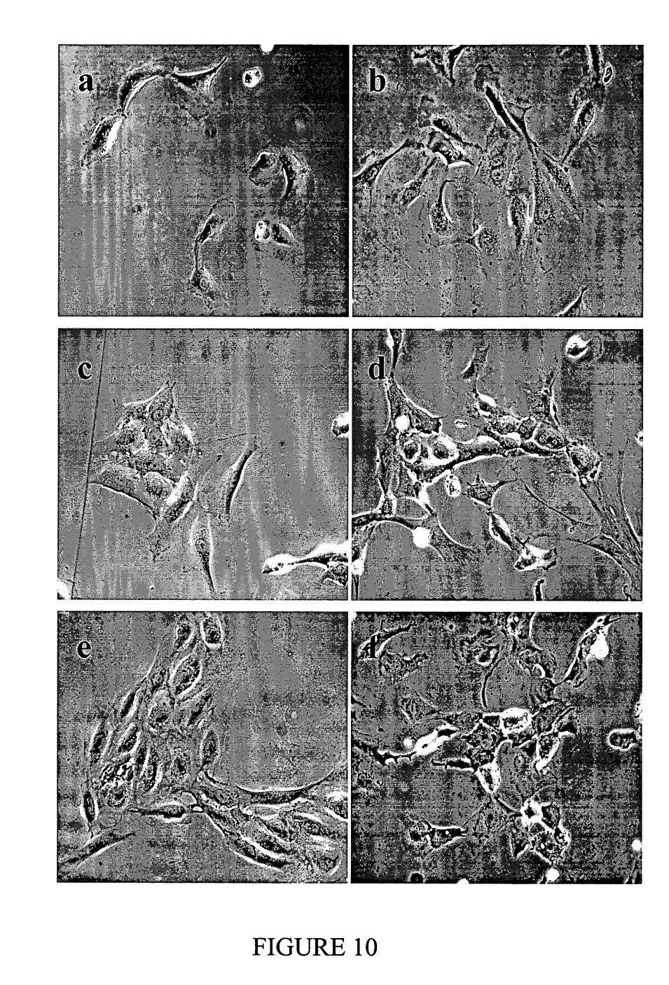

The morphology of the seeded cells was monitored microscopically every day. Cells seeded on plastic (FIG. 10a) appeared elongated and did not divide, indicating that their attachment to the substrate was not strong enough to promote cell spreading and division. Cells seeded on gelatin coating (FIG. 10b) appeared attached better and formed a confluent monolayer after 3 days. Cells seeded on HA coating (FIG. 10c) appeared elongated and attached, indicating that HA is not toxic as a substrate to endothelial cells. Part of the cells seeded in HA (FIG. 10d) appeared attached to the substrate, but many cells remained rounded and did not spread, indicating that floating in HA milieu could not provide a proper substrate for EC attachment and spreading. Cells seeded on gelatin and covered with HA after 24 hours (FIG. 10e) appeared attached and spread, indicating that HA was not toxic or harmful to the cells. Cells seeded on laminin coating (FIG. 10f) appeared round and did not attach well to the substrate. Debris of dead cells was apparent in the medium indicating that laminin coating or presence at this concentration is toxic to endothelium.

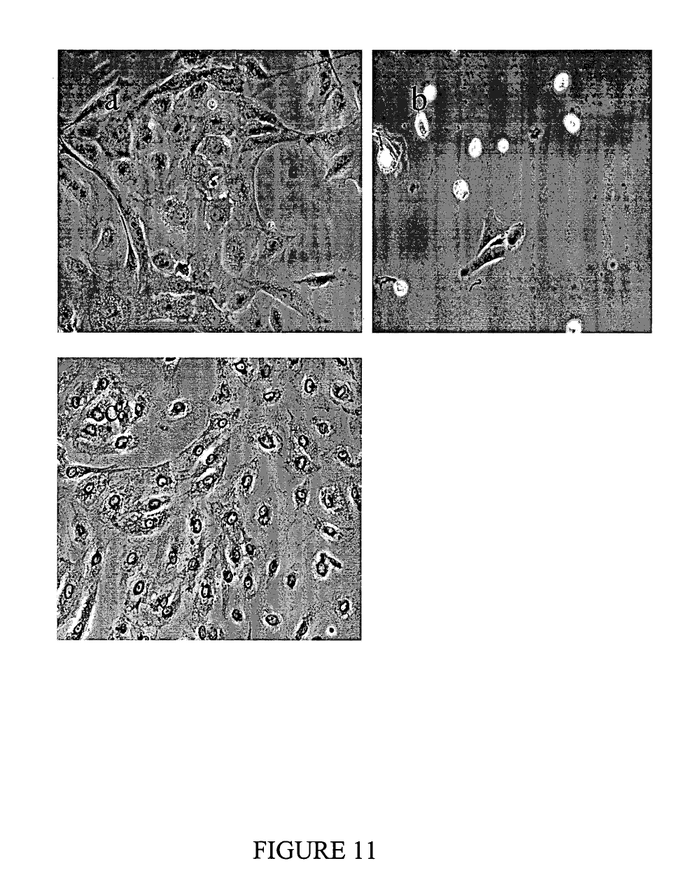

Cells seeded on HA-LN-gel coating (FIG. 11a) appeared flattened, viable and formed a typical polygonal monolayer, indicating that HA-LN-gel can serve as a suitable substrate for EC attachment and growth. Most of the cells seeded in HA-LN-gel (FIG. 11b) appeared rounded and floating, indicating that HA-LN-gel cannot serve as a milieu for EC. The cells that did not reach the bottom of the well remained round 48 hours after plating, and debris was apparent indicating cell death. Cells seeded on gelatin and after 24 hours covered with HA-LN-gel (FIG. 10c) appeared senescent, as if something was toxic either in the medium or in the coating.

The findings of the experiment described above indicated that either laminin alone or as a component of the HA-LN-gel, at the concentration examined, had some toxic effects on EC. Therefore the above experiment was repeated using 10% of the laminin concentration. Endothelial were seeded in 12 well plates in triplicates (50000 cell/well) as follows:
1. Cells on gelatin coating as control.
2. Cells on HA-LN-gel (10% of the usual concentration of laminin) coating.
3. Cells in HA-LN-gel (10% of the usual concentration of laminin)
4. Higher concentration of cells (100,000/well) in HA-LN-gel (10% of the usual concentration of laminin).

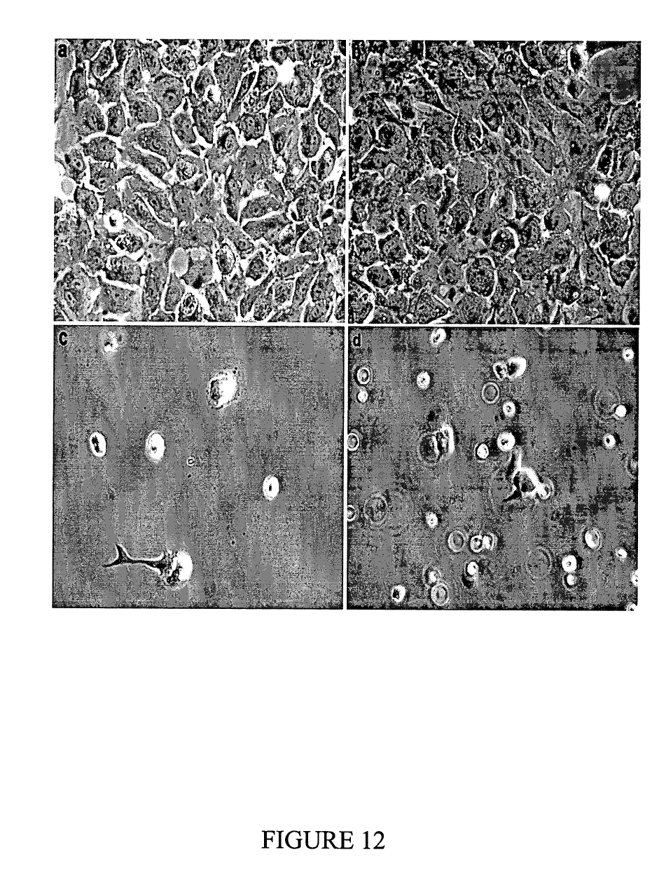

Endothelial cells seeded on gelatin or HA-LN-gel coatings (FIGS. 12a and b, respectively) attached and spread on the substrate and formed a confluent monolayer of viable polygonal cells. On the contrary, the majority of endothelial cells seeded in gel remained rounded and did not spread (FIGS. 12c and d). Only cells that sunk to the bottom of the well appeared spread. Cells that remained rounded appeared viable (glowing), but without attachment to a matrix cells death was observed within 78 hours.

In conclusion, HA-LN-gel is a suitable substrate for endothelial cells to grow on, however not in. These results show that composite implants made of HA-LN gels, as well as medical devises covered with the gel, can be successfully cover with endothelial cells to improve their function.

The foregoing examples of certain currently preferred embodiments are provided merely for illustrative purposes and are not to be construed as limitative. The scope of the invention is to be defined solely by the claims which follow.

### REFERENCES

Balazs E A, Denlinger J L (1988). In: The Biology of Hyaluronan, CIBA Foundation Symposium 143, pp. 265. Eds. Everd D, Whelan J. Wiley and Sons, Chichester, Sussex.

Condic M L, Letourneau P C (1997). Ligand-induced changes in integrin expression regulate neuronal adhesion and neurite outgrowth. Nature, 389, 852-856.

Colognato H, Yurchenco P D (2000) Form and function: the laminin family of heterotrimer. Dev. Dyn. 218, 213-234.

Gahwiler B H (1984) Experientia 40, 235.

Goldman S A, Nedergaard M, Crystal R G, Fraser R A R, Goodman R, Harrison-Restelli C,

Hunter D D, Cashman N, Morris-Valero R, Bulock J W, Adams S P, Sanes J R (1991). An LRE (leucine-arginine-glutamate) dependent mechanism for adhesion of neurons to S-laminin. J. Neurosci. 11, 3960-3971.

Ivins J K, Colognato H, Kreidberg J A, Yurchenco P D (1998) Neuronal receptors mediating responses to antibody activated laminin-I. J. Neurosci. 18, 9703-9715.

Jaffe et al (1972) Circulation 46, 211-253.

Jaffe et al (1973) J. Clin Invest 52, 2745-2756

Knudson C B, Knudson W (1993) FASEB. 7, 1233.

Kapfhammer J P, Schwab M E (1992). Cell Biol. 4, 863.

Laurent T C (1964) Biochem. Biophys. Acta. 93, 106.

Liesi P, Seppala I, Trenkner E (1992) Neuronal migration in cerebella microcultures is inhibited by antibodies against neurite outgrowth domain of laminin. J. Neurosci. Res. 33, 170-176.

Longaker M T, Chiu E S, Harrison M R, Crombleholme T M, Langer J C, Preston B, Davies M, Ogston A G (1965) Biochem. J. 96, 449.

Luckenbill and Edds, L (1997) Laminin and the mechanism of neuronal outgrowth. Brain Res. Rev. 23, 1-27.

Mecham R P (1991) Receptors for laminin on mammalian cells. FASEB J. 5, 2538-2546.

Miner J H, Patton B L (1999) Laminin II. Int. J. Biochem. Cell. Biol. 31, 811-816.

Powell S K, Kleinman H K (1997) Neuronal laminins and their cellular receptors. Int. J. Biochem. Cell Biol. 29, 401-414.

Robinson D, Halperin N, Nevo Z (1990) Calif. Tissue. Int. 46, 246.

Ruohslahti E (1988) Ann. Rev. Cell Biol. 4, 229.

Tao Peng et al (1996) Role of polymers in improving the results of stenting in coronary arteries. Biomaterials 17, pp. 685-694.

Thomas L, Eton T, Stamenkovic I, Mihm M C, Byers R (1993) J. Inves. Dermatol. 100, 115.

Toole B P (1976) In: Neural Recognition, p. 275. Ed. Barondes S H. Plenum Publishing Corporation, New York.

1. A biocompatible matrix comprising hyaluronic acid and laminin further comprising a bioactive compound or drug selected from the group consisting of a hormone, a growth factor, an antioxidant, a proteolytic enzyme, an anti-fibrotic agent, a chemotherapeutic anti-proliferative agent, a coagulative agent, an anti-coagulative agent, an immunomodulator, or a growth inhibitor.

2. The biocompatible matrix of claim 1 which is cross-linked by an exogenous cross-linking agent to form a combined gel.

3. The biocompatible matrix of claim 2 wherein the exogenous cross-linking agent is a sugar.

4. The biocompatible matrix of claim 1 wherein the gel has a viscosity of 4-48 centipoise.

5. The biocompatible matrix of claim 1 comprising 0.05% to 5% of hyaluronic acid.

6. The biocompatible matrix of claim 1 comprising 0.005% to 0.5% of laminin.

7. The biocompatible matrix of claim 1 wherein the growth factor is selected from the group consisting of brain-derived neurotrophic factors (BDNF), nerve growth factors (NGF), insulin-like growth factor-1 (IGF1), leukemia inhibitory factor (LIF), pifithrin-α and an antisense oligonucleotides of p53.

8. The biocompatible matrix of claim 1 wherein the antioxidant is selected from the group consisting of ascorbic acid, dehydroepiandrosterone (DHEA), melatonin, N-acetyl-L-cysteine and retinoic acid.

9. The biocompatible matrix of claim 1 further comprising a structural component selected from the group consisting of an extracellular matrix component, a natural polymer, a synthetic polymer, or a mixture thereof.

10. A cell culture comprising a plurality of cells other than cells of a neuronal explant cultured in or on a matrix comprising hyaluronic acid and laminin cross-linked to form a combined gel.

11. The cell culture of claim 10 comprising a plurality of cell types.

12. The cell culture of claim 10 comprising a cloned cell type.

13. The cell culture of claim 10 comprising a bioengineered type.

14. The cell culture of claim 10 comprising an autologous cell type.

15. The cell culture of claim 10 comprising stem cells.

16. The cell culture of claim 15 comprising an embryonic stem cell type.

17. The cell culture of claim 15 comprising an adult stem cell type.

18. The cell culture of claim 17 comprising a bone-marrow stem cell type.

19. The cell culture of claim 10 where the cells are cultured on the exposed surface of a combined hyaluronic acid laminin gel.

20. The cell culture of claim 19 comprising endothelial cell types.

21. An implant comprising a biocompatible matrix according to claim 1.

22. An implant comprising a cell culture according to claim 10.

23. A method for preparing a biocompatible matrix to be implanted in a human subject, which comprises:
hydrating a hyaluronic acid or salt or hyaluronan;
selecting a laminin solution; and
cross-linking the hydrated hyaluronan and laminin, with the optional addition bioactive or structural components, to form a combined gel.

24. The method of preparing the biocompatible matrix of claim 23 which further comprises shaping the matrix.

25. The method of claim 23 which further comprises culturing or embedding cells in or on the gel.

26. The method of claim 25 wherein the cultured cells are adherent on an exposed surface of the combined gel.

27. The method of claim 26 wherein the cultured cells are endothelial cells.

28. The method of claim 23 further comprises supplementing the gel with bioactive compound or drug selected from the group consisting of a hormone, a growth factor, an anti-oxidant, a proteolytic enzyme, an anti-fibrotic agent, a chemotherapeutic anti-proliferative agent, a coagulative agent, an anti-coagulative agent, an immunomodulator, or a growth inhibitor.

29. The method of claim 28 wherein the growth factor is selected from the group consisting of brain-derived neurotrophic factors (BDNF), nerve growth factors (NGF), insulin-like growth factor-1 (IGF1), leukemia inhibitor factor (LIF), pifithrin-α. and antisense oligonucleotides of p53.

30. The method of claim 27 wherein the antioxidant is selected from the group consisting of ascorbic acid, dehydroepiandrosterone (DHEA), melatonin, N-acetyl-L-cystein and retinoic acid.

31. A method for transplanting cells other than neuronal cells to an individual in need thereof, comprising the step of transplanting an implant comprising cells in or on a biocompatible matrix comprising hyaluronic acid and laminin cross-linked to form a combined gel.

32. A medical device comprising the biocompatible matrix of claim 1, and further comprising endothelial cells attached to an exposed surface of the gel.

33. The medical device of claim 32 wherein the biocompatible matrix forms a coating on the exposed surfaces of the device.

34. The medical device of claim 32 further comprising a bioactive compound or drug.

35. The medical device of claim 33 further comprising a bioactive compound or drug.
